(19) United States
(12) Patent Application Publication
(10) Pub. No.: US 2007/0167509 A1
(43) Pub. Date: Jul. 19, 2007

(54) Use of phenserine and analogs to treat behavioral problems and improve trainability

(76) Inventors: Joseph Araujo, Toronto (CA); William Milgram, Toronto (CA); Christa Studzinski, Toronto (CA); Nigel Greig, Phoenix, MD (US); Donald Ingram, Baton Rouge, LA (US)

Correspondence Address: DALINA LAW GROUP, P.C., 7910 IVANHOE AVE. #325, LA JOLLA, CA, 92037, US

(21) Appl. No.: 11/453,564

(22) Filed: Jun. 14, 2006

Related U.S. Application Data
(60) Provisional application No. 60/690,699, filed on Jun. 14, 2005.

(51) Int. Cl. A61K 31/407

(52) U.S. Cl. 514/411

(57) ABSTRACT

A method of treating behavioral problems, including those related to aging, and improving trainability in dogs is disclosed. The method involves administering phenserine, a phenserine analog, derivative or a metabolite thereof to a pet or service dog in need of such treatment.

This application claims benefit of U.S. Provisional Patent Application Ser. No. 60/690,699, filed Jun. 14, 2005 entitled “USE OF PHENSERINE AND ANALOGS TO TREAT BEHAVIORAL PROBLEMS AND IMPROVE TRAINABILITY” the specification of which is hereby incorporated herein by reference.

### BACKGROUND OF THE INVENTION

1. Field of the Invention

Embodiments of the invention described herein pertain to the field of drug treatments. More particularly, but not by way of limitation, embodiments of the invention are directed to the use of phenserine, its analogs and metabolites to treat behavioral problems, improve trainability and treat other cognitive dysfunctions in canines.

2. Description of the Related Art

Aged dogs show a progressive neurodegenerative disorder classified as Canine Cognitive Dysfunction Syndrome (CDS) (Landsberg and Ruehl, 1997; Bain et al., 2001; Ruehl et al., 1995). CDS is characterized by a decline in learning, memory, perception and awareness, which is manifest by behavioral problems including decreased exploration, sleep disturbances, housetraining deficits, restlessness, attentional difficulties, decreased motivation, alterations in grooming and increased anxiety. CDS impairs the quality of life of the dog, and also impacts the bond between the pet and master, thereby decreasing the master's enjoyment of the pet. The prevalence of CDS was indicated in a study in 180 dogs with no identifiable health problems (Neilson, et al., 2001). Twenty-eight percent of owners of 11-12 year-old dogs and 68% of owners of 11-12 year-old dogs reported at least one category consistent with CDS. Ten percent of owners of the 11-12 year old dogs and 36% of the owners of 15 to 16 year old dogs reported signs in 2 or more categories.

CDS also has problematic effects on service dogs, which are highly skilled dogs that are specially trained to carry out a uniquely important function. They include but are not limited to: seeing-eye dogs trained to help the blind; hearing dogs trained to help physically disabled individual; special skilled dogs trained on an individual basis to assist a person's special needs; and military working dogs trained for a variety of military functions. The Lyons foundation in Oakville, Ontario, Canada estimates that the cost to train a single dog is about $20,000. These dogs are typically retired between 7-8 years of age because they become less attentive and slow down. Thus, the cost of training dogs may be reduced first by counteracting the reduction in function because of the development of CDS, and second, by improving trainability. Improving trainability of dogs may also be applicable to the companion animal market, as companion dogs may also show training impairment at young ages possibly due to reduced attention.

Behavioral problems in dogs can be evaluated objectively using neuropsychological tests. The inventors have conducted several studies analyzing learning, memory and attentional processes of dogs. Initially, we reported that aged dogs performed more poorly than young dogs on visual-based neuropsychological tests (Milgram et al., 1994) and on a spatial memory test (Head et al., 1995). More recently, the inventors have shown that dogs that are older than 12 show widespread impairment in complex learning and retaining information for more than 10 s, suggesting that as dogs age an increasing proportion will develop cognitive impairment. Aged dogs are particularly susceptible to deficits in executive function (Tapp et al., 2003 b;Tapp et al., 2003a) and recent memory (Adams et al., 2000b; Chan et al., 2002) such that these deficits may occur much earlier than CDS is detected clinically.

These age-related alterations in behavior and cognition likely reflect neurodegenerative changes in the canine central nervous system (CNS). Aged dogs show cortical atrophy, particularly in the prefrontal cortex (Tapp et al., 2004), and a corresponding increase in the size of the lateral ventricles (Su et al., 1998). At a cellular level, distorted soma, loss of dendritic spines, shrinkage of dendritic branches and tortuous apical dendrites are seen. Increases in oxidative stress are also reported (Head et al., 2002).

Additionally, neuropathological deposits of beta-amyloid (AB) protein are present in the aged dog brain. The structure of this protein in the dog brain is identical to that found in aged humans suffering from Alzheimer's disease. The morphology of AB deposits is that of a diffuse subtype and the consequent plaques are thioflavin—S negative and therefore probably lack beta-pleated sheet formation (Cummings et al., 1993).

A direct link between AB pathology and dysfunction in learning and memory is established in the dog (Head et al., 1998). In particular, declarative-type tasks (visual discrimination, reversal and memory tasks), but not the procedural-type tasks (reward and object approach learning) were strongly correlated with AB deposition in both the prefrontal and entorhinal cortices.

In view of the foregoing, there is a need in the art for a treatment for behavioral problems in dogs, which may be accomplished by the use of cholinesterase inhibitors, which augment cholinergic neurotransmission.

### BRIEF SUMMARY OF THE INVENTION

One or more embodiments of the invention are directed to the use of phenserine, a cholinesterase inhibitor, for treating behavioral problems associated with cognitive dysfunction in dogs or other companion mammals. Accordingly, one or more embodiments of the invention comprise a method of treating both age-related and non-age-related behavioral problems in dogs by administering an effective amount of phenserine or an analog, derivative or metabolite of phenserine to a dog in need thereof. To further enhance or supplement the treatment additional interventions may be combined with phenserine.

Behavioral problems that may be treated according to embodiments of the invention include, but are not limited to, learning, memory, attention and age-related neurological disorders, such as CDS and signs associated with CDS. Overall, treatment with phenserine, its analogs and its metabolites improves the overall quality of life for both the companion animal such as a dog and its owner.

Other features and advantages of embodiments of the invention will become apparent from the following detailed description. It should be understood, however, that the detailed description, and the specific examples, while indicating preferred embodiments of the invention, are given by way of illustration only, since various changes and modifications within the spirit and scope of the invention will become apparent to those skilled in the art from this detailed description. The claims however, and the full scope of their equivalents are what define the metes and bounds of the invention.

### BRIEF DESCRIPTION OF THE DRAWINGS

The invention will now be described in relation to the drawings in which:

FIG. 1 shows mean performance accuracy on a task of recognition memory of phenserine and control groups on blocks of 5 test sessions. Dogs on phenserine showed improved performance on the last block vs. the first. Error bars represent the standard error.

FIG. 2 depicts acquisition errors on the a spatial memory task for animals on phenserine (label 1) and animals on placebo (label 2).

FIG. 3 shows the effects of saline (SAL) and scopolamine (SCP) administration on spatial memory performance accuracy at 20- and 80-s delays for animals receiving placebo and phenserine. In the placebo group, performance deteriorated with either increased delay or scopolamine treatment. Phenserine facilitated performance at the long delay under the saline condition. In scopolamine-treated dogs, phenserine provided protection at the short delay. Error bars represent the standard error.

FIG. 4 depicts the effects of saline (SAL) and scopolamine (SCP) administration on spatial memory performance accuracy at 20- and 80-s delays in dogs previously treated with placebo or phenserine. The results demonstrate that prior phenserine treatment provided long-lasting protection against scopolamine induced impairment. Error bars represent the standard error.

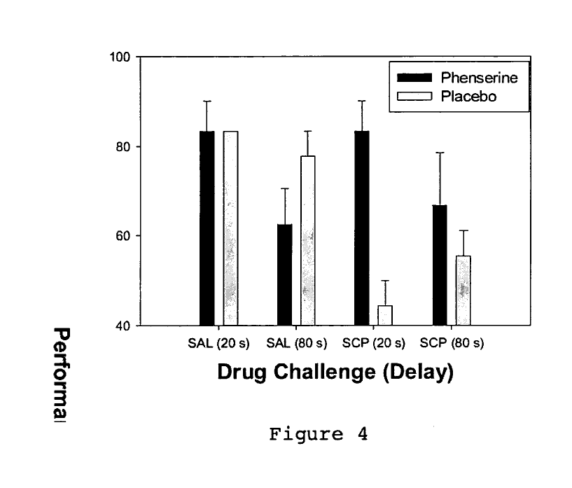

FIG. 5 depicts spatial memory performance accuracy of the placebo group during administration of placebo and after placebo discontinuation under saline (SAL) and scopolamine (SCP) challenge. Performance accuracy of the placebo group did not differ at any delay under any challenge when placebo was discontinued. Error bars represent the standard error.

FIG. 6 shows spatial memory performance accuracy of the phenserine group during administration of phenserine and after phenserine discontinuation under saline (SAL) and scopolamine (SCP) challenge. Performance accuracy of the phenserine group decreased at the 80-s delay under the saline condition after phenserine was discontinued, however, performance was maintained at the 20-s delay under scopolamine when phenserine was discontinued. Error bars represent the standard error.

FIG. 7 is a graph showing errors-to-criterion for animals learning progressively more difficult oddity discrimination tasks. Animals performed more poorly on the more difficult task, ODD2, than ODD 1. Compared to control subjects, animals treated with phenserine did not differ on ODD1, but did commit fewer errors on ODD2. Error bars represent the standard error.

### DETAILED DESCRIPTION OF THE INVENTION

One or more embodiments of the invention relate to a method for treating behavioral problems. More particularly, but not by way of limitation, those associated with age, in dogs comprising administering an effective amount of phenserine or an analog, derivative or metabolite of phenserine to a dog in need thereof. Embodiments of the invention also relates to a method of treating age-related neurodegeneration in dogs by administering an effective amount of phenserine or an analog, derivative or metabolite of phenserine to a dog in need thereof. Readers should note that although dogs are referred to throughout this specification for purposes of example, one or more embodiments of the invention are applicable to other mammals. For instance, the method for treating behavioral problems described herein in the context of dog also has applicability to companion animals other than dogs.

Phenserine is a selective acetylcolinesterase inhibitor, well described in references provided herein. The chemical name of phenserine is (−)-phenylcarbamoyleseroline. In addition to acetylcolinesterase inhibition, phenserine also demonstrates an effect on AB processing, particularly by reducing levels of APP.

The term “analog of phenserine” typically means any compound that shares structural similarity with phenserine (although one or more functional groups may be substituted with similar functional groups) and is useful in treating behavioral problems in dogs.

The term “derivative of phenserine” generally refers to any compound that is derived from phenserine and is useful in treating behavioral problems in canines. This includes a compound where a functional group is chemically derived.

The term “metabolite of phenserine” includes, but is not limited to, any compound that is produced by the metabolism of phenserine and that is useful in treating behavioral problems in dogs.

The term “phenserine or an analog, derivative or metabolite of phenserine” includes but is not limited to pharmaceutically acceptable salts or derivatives of phenserine or its analogs, derivatives or metabolites. Examples of preferred formulations of phenserine, its analogs, and derivatives are included in references herein. The process for producing phenserine and its analog are further described in U.S. Pat. No. 6,495,700.

The term “behavioral problem” generally means, but is not limited to, any problem that arises from the normal course of aging in a dog, or that occurs throughout the lifespan of a dog. Examples of behavioral problems that may be treated according to the present invention include, but are not limited to, learning, urinary incontinence, bladder control, soiling, alertness, attention, exploratory behavior, memory and age-related neurological disorders. Some examples of the type of age related disorders one or more embodiments of the invention are able to counteract include but are not limited to a companion animal's general trainability, learning capacity, aggressiveness or other problems associated with the animal's inability to remember information.

The quantities referenced herein are approximations and readers should note that any quantity that accomplishes the same effect described herein are within the scope and spirit of the invention. Dose quantities can be reasonably varied so long as the quantity does not exceed a level of toxicity tolerable to the subject under treatment.

As is shown in the examples described herein embodiment of the invention show that phenserine has the following beneficial effects on dogs and other companion animals.

1. Improved Trainability in Non-Demented Dogs

When adult dogs are administered a daily dose of phenserine (e.g.,0.5 mg/kg) the dogs experience an improvement in learning and respond correctly on an object recognition memory task, the delayed-non-matching-to sample task, compared to placebo controls. This suggests that phenserine can improve trainability in dogs that are not considered aged and that likely do not exhibit AB neuropathology.

2. Improved Learning in Demented Dogs

Administering phenserine at a dose (e.g., 0.5 mg/kg) decreases the amount of time required to learn complex rules and thereby reverses age-dependent cognitive deterioration. The inventors demonstrated this using 2 complex tasks, a delayed-non-matching-to-position task and an oddity discrimination task, which are both sensitive to canine aging deficits.

3. Improved Memory in Demented Dogs

As described herein it has been discovered that phenserine can counteract, or reverse age-dependent deterioration in memory because dogs treated with a dose of phensire (e.g., 0.5 mg/kg) do not demonstrate the delay-dependent deficits seen in placebo-treated dogs.

Furthermore, the inventors demonstrated that phenserine attenuates deficits induced by the anti-cholinergic drug scopolamine, suggesting that phenserine can reverse age-dependent cholinergic memory deficits in aged dogs.

The learning and memory improvement caused by phenserine is not only useful for dogs that are pets but also for service dogs. Phenserine can prolong the productive lifespan of service dogs by improving their attention and working memory capacity as well as by improving trainability.

4. Age-Related Neurological Disorders

Maintenance of aged dogs on phenserine will retard the development of both age dependent neuropathology and neurological disorders.

For all of the above indications, phenserine, its analog, derivative or metabolite is administered at dosages and for periods of time necessary to achieve the desired result (referred to herein as an “effective amount”). Preferably, the phenserine its analog, derivative or metabolite is administered in an amount from about 0.01 to about 1 mg/kg, preferably from greater than 0.1 mg/kg to about 1 mg/kg and more preferably from about 0.3 to about 0.7 mg/kg.

The phenserine its analog, derivative or metabolite preferably is administered orally by way of a tablet, capsule or solution. The phenserine its analog, derivative or metabolite also can be administered through any other suitable route such as parenterally, intravenously, subcutaneously, intramuscularly, transdermally, intranasally, rectally, or by inhalation.

Phenserine, its analog, derivative or metabolite can be obtained from commercial sources or can be synthesized according to references provided herein. Once synthesized, phenserine, its analog, derivative or metabolite can be formulated into a pharmaceutical composition suitable for administration to dogs. The composition can be prepared by known methods for the preparation of pharmaceutically acceptable compositions, which can be administered to dogs, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., U.S.A. 1985) On this basis, the compositions include, albeit not exclusively, solutions of the substances in association with one or more pharmaceutically acceptable vehicles or diluents, and contained in buffered solutions with a suitable pH and iso-osmotic osmotic with the physiological fluids.

### EXAMPLES

The present examples are intended to demonstrate the effectiveness of phenserine, its analogs and metabolites thereof, on canine cognitive function. Previously it was shown that performance on the described tests is correlated with clinical measures of clinically relevant behavioral improvements (Siwak et al., 2001). Three examples are provided in which the effects of phenserine on cognitive function in dogs is demonstrated. In the first example, the effects of phenserine on recognition memory performance are examined in middle-aged dogs. The second example demonstrates the effect of phenserine on visuospatial relearning, memory performance, and scopolamine-induced performance deficits in senior dogs. The final example demonstrates the effects of phenserine on learning a series of progressively more difficult oddity discrimination problems.

### Example 1

### The Effects of Phenserine on Recognition Memory in Middle-Aged Dogs.

The present study assessed the effects of a 0.5 mg/kg dose of phenserine in middle-aged dogs tested on a recognition memory task.

Six middle-aged beagle dogs (4 female and 2 male) served as subjects. Dogs were divided equally into 2 groups matched for performance on their most recent 10 sessions on the delayed-non-matching-to-sample task (DNMS; (Callahan et al., 2000). One group was administered phenserine (0.5 mg/kg) via capsule in a meatball and the second group served as control. Animals were tested one-hour after drug administration and were tested on a total of 20 test sessions.

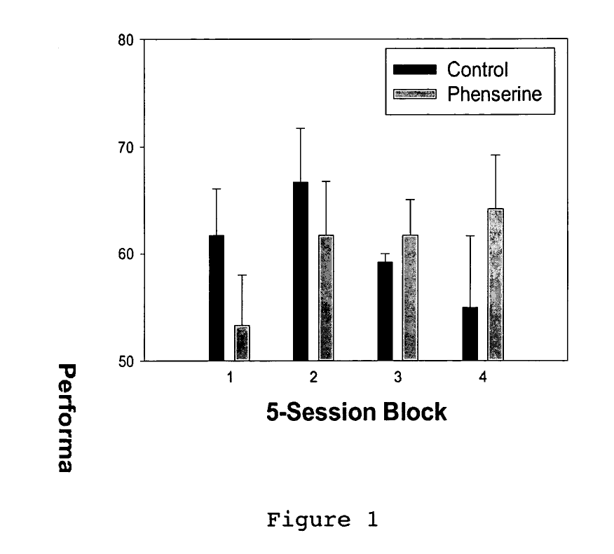

To examine the effects of phenserine on DNMS performance, mean performance over groups of five test sessions was analyzed (see e.g., FIG. 1). The subjects receiving phenserine showed a trend for improved performance in the last 5-session block (p=0.074) compared to the first; the performance of the control subjects, by contrast, showed some deterioration.

As FIG. 1 illustrates these results demonstrate a clear trend for improved performance in subjects treated with phenserine, which suggests that phenserine is able to improve learning ability or trainability in subjects not considered old or demented. No obvious adverse effects of phenserine were seen using the 0.5 mg/kg dose.

### Example 2

### The Effects of Phenserine on Visuospatial Memory and Learning in Senior Dogs

The purpose of the present example is to demonstrate the effects of phenserine on the performance of senescent beagle dogs on a delayed-non-matching-to-position task (DNMP), which assesses visuospatial finction and memory (Adams et al., 2000b; Chan et al., 2002) and deteriorates at an early age in dogs (Adams et al., 2000). What follows is a demonstration of the effects of phenserine on relearning the task and on scopolamine-induced deficits.

Five male and two female senior beagle dogs (M+S.D.=14.60+1.46 years) served as subjects (Table 1). Dogs were divided into two groups matched on their most recent DNMP performance; the first group (N=4) was administered phenserine (0.5 mg/kg) via capsule in a meatball and the second group (N=3) was administered a placebo meatball. Dogs received a five-day wash-in period prior to study initiation and were administered their respective treatments 1-hour prior to DNMP testing. Dogs were trained initially on the DNMP using a 5-s delay until a 2-stage criterion was passed. The first stage required animals to achieve a score of 11/12 on one day, a score of 10/12 over two consecutive days, or a score of 29/36 over three consecutive days. Subsequently, the dogs were required to achieve a score of 26/36 over the following three days to pass the second stage.

TABLE 1
Subjects in the DNMP study. Date of birth, age at the
initiation of the study and drug group is indicated.

| Subject | Recorded Date of Birth | Age at Initiation | Treatment Group |
|---|---|---|---|
| Henry | 05 May 1987 | 16.37 | Phenserine |
| Odo | 04 Aug. 1988 | 15.12 | Placebo |
| Quark | 04 Aug. 1989 | 14.12 | Phenserine |
| Seven | 04 Aug. 1989 | 14.12 | Phenserine |
| Peanut | 18 Sep. 1987 | 12.00 | Phenserine |
| Jooky | 01 Sep. 1987 | 16.04 | Placebo |
| Piggy | 02 Apr. 1989 | 14.46 | Placebo |

Following relearning of the DNMP, dogs were tested on a variable-delay paradigm in which delays of 20 and 80 s were used. The delays occurred equally among the trials in a daily test session. We have previously shown that the 15-μg/kg dose selectively impairs DNMP performance (Araujo et al., 2004a). The experimental design is shown in Table 2. The dogs were first divided into two groups, one that was administered phenserine and the other received a placebo control. All subjects were given a 5-day stabilization period and then tested under scopolamine (15 μg/kg, SC) and saline (SC), administered 45 min prior to testing, over two test days separated by a treatment washout day. One day following the second treatment day, animals were removed from phenserine or placebo treatments and re-tested on the stabilization days and under scopolamine and saline treatment as indicated above.

TABLE 2
Experimental Design. There were two groups, one initially treated
with phenserine the other received a placebo control. Both groups
were tested under both saline and scopolamine treatments. Following
the first scopolamine challenge, all subjects were discontinued
from phenserine and placebo treatment.

| | Phenserine Group | | Placebo Group | |
|---|---|---|---|---|
| Test | Order 1 | Order 2 | Order 1 | Order 2 |
| Day | (N = 2) | (N = 2) | (N = 1) | (N = 2) |
| 1-5 | Stabilization | | | |
| 6 | Scopolamine | Saline | Scopolamine | Saline |
| 7 | Washout | | | |
| 8 | Saline | Scopolamine | Saline | Scopolamine |
| 9 | Washout - Phenserine/Placebo Discontinued | | | |
| 10-15 | Stabilization | | | |
| 16 | Scopolamine | Saline | Scopolamine | Saline |
| 17 | Washout | | | |
| 18 | Saline | Scopolamine | Saline | Scopolamine |

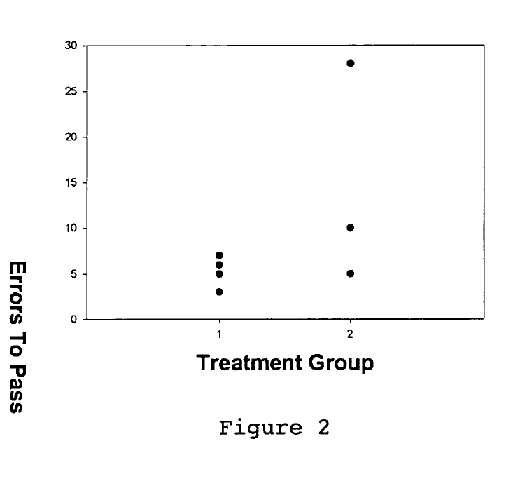

The subjects administered phenserine committed fewer mean errors to relearn the DNMP (M+SEM=5.25+0.85) than subjects administered the placebo (M+SEM=14.33+6.98), (see e.g., FIG. 2). Furthermore, when compared to the most previous relearning rates, the phenserine group tended to show fewer errors (M+SEM=5.00+4.51) and the placebo group more errors (M+SEM=6.33+9.53).

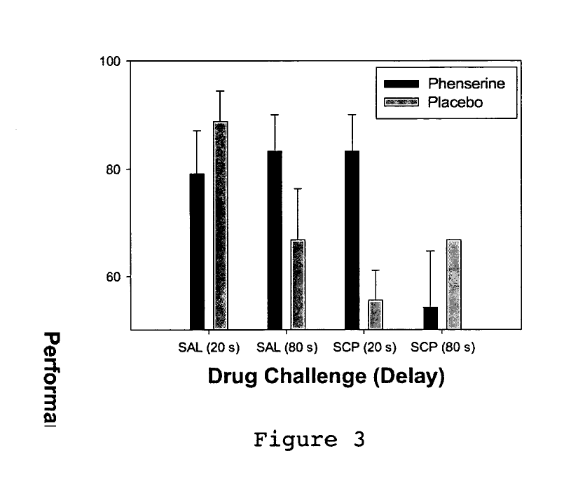

The results of the drug challenge experiment are depicted in FIG. 3. The placebo group demonstrated a performance deficit under the saline condition with increasing delay (p=0.081). After treatment with scopolamine, the placebo group showed performance deficits at both the 20- (p=0.038) and 80-s (p=0.082) delays. The phenserine group, by contrast, showed decreased performance at the 80-s delay after treatment with scopolamine, but not after treatment with saline. Comparisons between the phenserine and placebo group revealed that phenserine attenuated the scopolamine-induced performance deficit at the 20-s delay [T(5)=2.99; p=0.031].

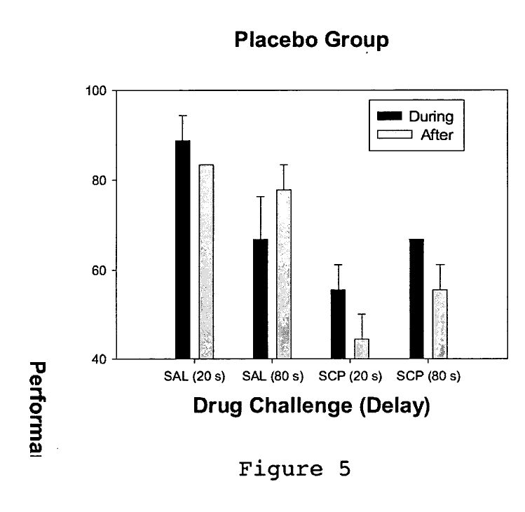

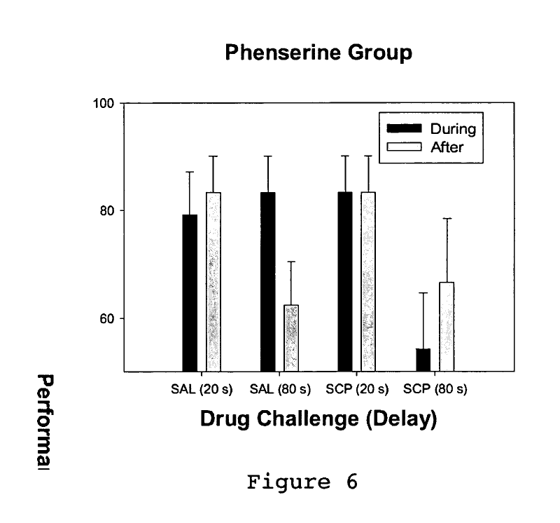

After phenserine treatment was terminated, a main effect of delay was found [F(1,3)=562.5; p=0.0061], which was due to decreased performance accuracy at the 80-s delay compared to the 20-s delay (p=0.014). Animals previously treated with placebo demonstrated a performance deficit under the saline condition with increasing delay and after treatment with scopolamine, which did not differ from the previous experiment (FIG. 5). The subjects previously receiving phenserine did not show a scopolamine-induced performance deficit at the 20-s delay [T(5)=4.18; p=0.0086], but did show delay-dependent deficits [T(4)=5.00; p=0.015] (FIG. 6).

Thus, the relearning data demonstrates that phenserine improves learning and trainability. The scopolamine experiment shows that phenserine is effective in preventing scopolamine from disrupting memory. In the initial scopolamine challenge experiment, the performance of the placebo group was identical to what we have seen previously. In particular, placebo dogs showed decreased performance accuracy with increasing delay and dose-independent performance deficits following scopolamine administration (Araujo et al., 2004a; Araujo et al., 2004). By contrast, the dogs that were administered phenserine demonstrated an unexpected pattern of performance. First, the phenserine group was not impaired at the 80- vs. 20-s delay under the saline condition, which suggests that phenserine improves memory performance. Further evidence to support this hypothesis was the finding that animals discontinued from phenserine subsequently demonstrated delay-dependent impairment under the saline condition. Second, the phenserine group did not show the characteristic scopolamine deficits that we would expect at 20 s; dogs of similar age showed significant impairment at a 5-s delay under scopolamine in a previous experiment. This study also showed that of the positive actions of phenserine continued even after phenserine treatment stopped. Thus, phenserine has long-lasting effects on memory.

Collectively, the present results suggest that phenserine improves visuospatial function and memory, and should be useful for the treatment of canine cognitive dysfunction.

### Example3

### The Effects of Phenserine on Oddity Discrimination Learning

The purpose of the present example is to demonstrate the effects of phenserine on learning of discrimination problems of increasing difficulty, and thereby to show that phenserine can improve trainability.

Methods: Eleven aged beagle dogs (M+S.D.=8.76+0.96 years) served as subjects. Animals were divided into 2 groups matched for previous performance on discrimination tests. One group received phenserine; and the other, placebo during the first oddity problem. For the second oddity problem, the treatments were crossed-over such that the animals given phenserine on the first task were now given a placebo and vice versa. Subjects received 0.5 mg/kg of phenserine in a capsule placed in a meatball or the placebo, which consisted of an empty capsule in a meatball, 1 hour prior to testing. Prior to each oddity level, subjects underwent a 5-day wash-in period.

The subjects were first given a preference test in which they were required to choose one of two objects. For oddity testing, the object chosen most often during the preference test, considered the preferred object, was never rewarded and appeared in duplicate. The object chosen least during the preference test was always rewarded. Thus, each trial consisted of a presentation of three objects; two identical preferred objects, and the rewarded object. All dogs were tested until they passed an a priori criterion, or were tested on 480 trials, whichever occurred first. All dogs were tested initially on an oddity discrimination (ODD1) that we presumed would be easier based on object differences, followed by a second, and presumably more difficult, problem (ODD2).

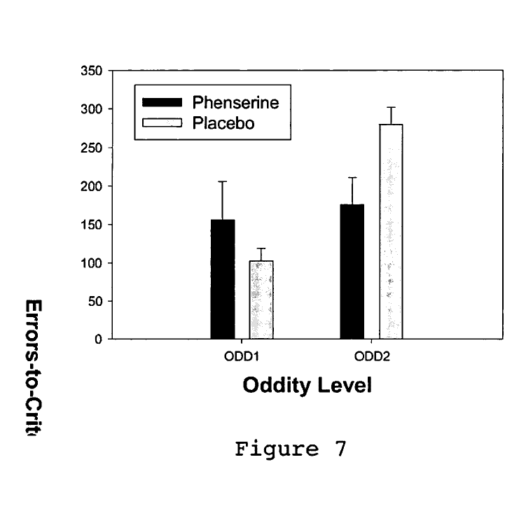

Results: To analyze the data, a 2-way repeated-measures ANOVA was conducted with drug treatment (phenserine vs. placebo) serving as a within-subject measure and order of treatments between oddity levels as a between-subject variable. A significant interaction between drug treatment and the order of treatment between levels was found [F(1,9)=14.20; p=0.0044]. The data are depicted in FIG. 7. No differences between phenserine and placebo groups were found on ODD1. For the placebo groups, an increase in errors occurred in ODD2 compared to ODD1 [T(9)=6.52; p=0.0001], which supported the presumption that ODD2 was the more difficult of the two problems. No differences were found between the phenserine groups on ODD1 and ODD2. On ODD2, however, dogs on placebo committed significantly more errors than dogs on phenserine [T(9)=2.39; p=0.04].

Discussion and Conclusions: In the present study, we used progressively more difficult complex discriminations to assess the effects of phenserine on learning. Aged dogs typically show an increase in errors to learn the oddity task when the difficulty of the task is increased (Milgram et al., 2002). The present study confirmed these results since animals on the control treatment committed significantly more errors on ODD2, which used objects that were more similar in appearance than ODD 1.

On ODD1, no differences were found between the phenserine and control groups. When task difficulty was increased, however, phenserine-treated subjects committed significantly fewer errors than the control group. These findings indicate that phenserine can attenuate age-dependent learning deficits. The implication of these findings is that phenserine will be useful as an agent for treating canine cognitive dysfunction, and also may be useful in treating other behavioral canine disorders and to reduce the training time required for dogs in the dog-service industry.

It is also beneficial and further enhances the effectiveness and/or palatability to phenserine-treated subjects when the phenserine methodology described herein is combined with other interventions. Some examples of the various types of interventions that provide increased benefits when combined with phenserine include, but are not limited to the following:
Nutritional Interventions—vitamins, supplements, phospholipids, antioxidants, and mitochondrial cofactors—Vitamin C (ascorbic acid; Stay—C), E, dl-lipoic acid, 1-carnitine, dl-alpha tocopherol, taurine, docosahexaenoic acid (DHA), omega-3 polyunsaturated fatty acid, omega-6 polyunsaturated fatty acid, proanthocyanins, anthocyanins, bioflavenoids, derivatives of glutathione, melatonin, ginkgo biloba (extract), ginseng (extract), bilberry (extract), blueberry (extract), fish oil, phosphatidylserine, phosphatidylcholine.
Metabolic Interventions—Those that provide alternate energy sources—inducers of ketosis—medium chain triglycerides—caprilic acid, Neobee™ 895.
Cholinergic modulators—M1 and M4 agonists/partial agonists, M2 antagonists, choline and derivatives, acetylcholinesterase inhibitors, nicotinic agonists/partial agonists—donepexil, rivastigmine, galantamine, huperzine A, milameline, MKC-231, nefiracetam, NS2330, tacrine, ispronicline
Serotonergic modulators—5HT4 agonists/partial agonists
Glutaminergic modulators—AMPA agonists/partial agonists (ampakines), NDMA antagonists/agonists/partial agonists—ampalex, memantine, neramexane
Dopaminergic modulators—1—derenyl, adrafanil, nicergoline
Amyloid—beta modulators—APP transcription inhibitors, immunotherapy, gamma- and beta secretase inhibitors, amloid-beta fibrillization inhibitors, alpha-secretase enhancers/promotors
Chelators—zinc, copper, aluminium and iron chelators—clioquinol
Calcium signal modulators—CREB agonists
Statins—lovastatin, pravastatin
Growth Factors—Cerebrolysin, neotrofin, CERE-110
Anti-inflammatory—NSAIDS, COX2 inhibitors—avlosulfon, ibuprofen, naproxen, rofecoxib, celocoxib, Carprofen
Sex-hormone modulators—estrogen and testosterone modulators—leuprolide, premarin
Anxiolytics (separation disorders tricyclic antidepressants, SSRIs, 5HT1 agonists, benzodiazapenes, antipsychotics—clomimpramine, amitrytaline, fluoxetine, buspirone, alprazolam, oxazepam, lorazepam, clonazepam, 1—deprenyl, acepromazine

It is also with the scope and spirit of the invention to create Phenserine-based carbamates. A exemplary formula of a chemical structure for phenserine-based carbamates follows.

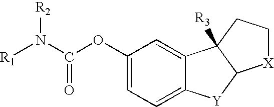

In one aspect, described herein are compounds having the formula I. Wherein each R₁ is, independently, hydrogen, a branched- or straight-chain alkyl group, a substituted or unsubstituted aryl group, a substituted or unsubstituted aralkyl group, an ether group, a carboxylate group, or an amide group, R₂ is, independently, hydrogen, a branched- or straight-chain alkyl group, or a substituted or unsubstituted aryl group, R₃ is, independently, hydrogen, a branched- or straight-chain alkyl group, or a substituted or unsubstituted aryl group, Y is, independently, O, S or N, and can be NR₄, where R₄ can independently be hydrogen, a branched- or straight-chain alkyl group, a substituted or unsubstituted aryl group, a substituted or unsubstituted aralkyl group, an ether group, a carboxylate group, or an amide group, X is, independently, O, S or N, and can be NR₅, where Rᵣ can independently be hydrogen, a branched- or straight-chain alkyl group, a substituted or unsubstituted aryl group, a substituted or unsubstituted aralkyl group, an ether group, a carboxylate group, or an amide group, and wherein the compound having the formula I is the substantially pure (−)-enantiomer, the substantially pure (+)-enantiomer, or a racemic mixture of the (−)-enantiomer and (+)-enantiomer.

Variables such as R₁−R₅, X, and Y used throughout the application are the same variables as previously defined unless stated to the contrary. The term “substantially pure” with respect to enantiopurity refers to greater than 95%, greater than 97%, greater than 98%, greater than 99%, greater than 99.5%, or 100% of one enantiomer with respect to the other enantiomer. The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Examples of longer chain alkyl groups include, but are not limited to, an oleate group or a palmitate group. A “lower alkyl” group is an alkyl group containing from one to six carbon atoms. The term “alkenyl group” as used herein is a hydrocarbon group of from 2 to 24 carbon atoms and structural formula containing at least one carbon-carbon double bond. Asymmetric structures such as (AB)C=C(CD) are intended to include both the E and Z isomers. This may be presumed in structural formulae herein wherein an asymmetric alkene is present, or it may be explicitly indicated by the bond symbol C.

The term “alkynyl group” as used herein is a hydrocarbon group of 2 to 24 carbon atoms and a structural formula containing at least one carbon-carbon triple bond. The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy. The term “cycloalkyl group” as used herein is a non-aromatic carbon-based ring composed of at least three carbon atoms.

Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus. The term “aralkyl” as used herein is an aryl group having an alkyl, alkynyl, or alkenyl group as defined above attached to the aromatic group. An example of an aralkyl group is a benzyl group. The term “ether” as used herein is represented by the formula ROR′, where R and R′ can be, independently, an alkyl, alkenyl, alkynyl, aryl, heteroaryl, cycloalkyl, cycloalkenyl, heterocycloalkyl, or heterocycloalkenyl group described above. The term “carboxylate” as used herein is represented by the formula —C(O)OH or the ester thereof.

Ester derivatives are typically prepared as precursors to the acid form of the compounds and accordingly can serve as prodrugs. Generally, these derivatives will be lower alkyl esters such as methyl, ethyl, and the like. Amide derivatives —(CO)NH₂, —(CO)NHR and —(CO)NR₂, where R is an alkyl group defined above, can be prepared by reaction of the carboxylic acid-containing compound with ammonia or a substituted amine.

It is contemplated that the pharmaceutically-acceptable salts or esters of the compounds described herein can be used as prodrugs or precursors to the active compound prior to the administration. For example, if the active compound is unstable, it can be prepared as its salts form in order to increase stability. Prior to administration, the salt can be converted to the active form. For example, the salt can be added to a saline solution to produce the active compound, followed by administration of the saline solution containing the active compound to the subject.

The term “amide” as used herein is represented by the formula —C(O)NR, where R can alkyl, alkenyl, alkynyl, aryl, heteroaryl, cycloalkyl, cycloalkenyl, heterocycloalkyl, or heterocycloalkenyl group described above. By “subject” is meant an individual. Preferably, the subject is a mammal such as a dog, but can include domesticated animals, such as cats, monkeys, etc., livestock (e.g., cattle, horses, pigs, sheep, goats, etc.), and laboratory animals (e.g., mouse, rabbit, rat, By “effective amount” is meant a therapeutic amount needed to achieve the desired result or results, e.g., inhibiting enzymatic activity. Herein, “inhibition” or “inhibiting” means to reduce activity as compared to a control. It is understood that inhibition can mean a slight reduction in activity to the complete ablation of all activity. An “inhibitor” can be anything that reduces the targeted activity.

Not all cholinesterase inhibitors have proven effective in reversing cognitive deficits in dogs and prior attempts to use such cholinesterase inhibitors have been ineffective. For instance, in a study to examine the effects of galantamine on cognitive performance in aged beagle dogs, tests of 10 aged dogs at doses of 2.5, 5 and 10 mg following scopolamine treatment. Galantamine was unable to reverse the scopolamine deficits.

In a second study, using a counter-balanced within-subject design, the effect CP-118,954, a benzylpiperidine, on DNMP performance in aged dogs showed no significant improvements. Using a counter-balanced within-subject design, we found no significant improvement on DNMP performance.

Classification of Acetylcholinesterase Inhibitors.

1. Carbamates—physostigmine, rivastigmine, phenserine, epastigmine

2. Aminoacridines—tacrine, velnacrine, suronacrine, ipidacrine, SM-10888

3. Benzylpiperidines—donepezil, CP-118,954, TAK-147

4. Phenanthrene alkaloid—galantamine

It is possible to accomplish the effects discussed herein and within the scope and spirit of the invention to use carbamates generally, and then more specifically phenserine and analogues. Hence the approach described is not limited solely to phenserine and its analogs, but includes carbamates generally.

All publications referenced below are herein incorporated by reference in their entirety to the same extent as if each individual publication, patent or patent application was specifically and individually indicated to be incorporated by reference in its entirety.

Adams B, Chan A, Callahan H, Siwak C, Tapp D, Ikeda-Douglas C, Atkinson P, Head E, Cotman C W, Milgram N W (2000) Use of a delayed non-matching to position task to model age-dependent cognitive decline in the dog. Behavioural Brain Research 108: 47-56.

Araujo J A, Chan A D F, Winka L L, Seymour P A, and Milgram N W. (2004a, In Press) Dose-specific effects of scopolamine on canine cognition: Impairment of visuospatial working memory, but not visuospatial discrimination. Psychopharmacology. Araujo J A, Tapp P D, Studzinski C M, Chan A D F, and Milgram N W. (2004b, In submission) Age sensitivity to scopolamine-induced working memory performance impairment in dogs.

Araujo J A, Studzinski C M, Milgram N W. (2004c, In submission) Further evidence for the cholinergic hypothesis of aging and dementia from the canine model of aging.

Bain M J, Hart B L, Cliff K D, Ruehl W W (2001) Predicting behavioral changes associated with age-related cognitive impairment in dogs. Journal of the American Veterinary Medical Association 218: 1792-1795.

Bartus R T, Dean R L, III, Beer B, Lippa A S (1982) The cholinergic hypothesis of geriatric memory dysfunction. Science 217: 408-414.

Callahan H, Ikeda-Douglas C, Head E, Cotman C W, Milgram N W (2000) Development of a protocol for studying object recognition memory in the dog. Progress in Neuro-Psychopharmacology & Biological Psychiatry 24: 693-707.

Chan A D, Nippak P M, Murphey H, Ikeda-Douglas C J, Muggenburg B, Head E, Cotman C W, Milgram N W (2002) Visuospatial impairments in aged canines (Canis familiaris): the role of cognitive-behavioral flexibility. Behavioral Neuroscience 116: 443-454.

Cummings B J, Su J H, Cotman C W, White R, Russell M J (1993) Beta-amyloid accumulation in aged canine brain: a model of early plaque formation in Alzheimer's disease. Neurobiology of Aging 14: 547-560.

Head E, Callahan H, Muggenburg B A, Cotman C W, Milgram N W (1998) Visual-discrimination learning ability and beta-amyloid accumulation in the dog. Neurobiology of Aging 19: 415-425.

Head E, Liu J, Hagen T M, Muggenburg B A, Milgram N W, Ames B N, Cotman C W (2002) Oxidative damage increases with age in a canine model of human brain aging. Journal of Neurochemistry 82: 375-381.

Head E, Mehta R, Hartley J, Kameka M, Cummings B J, Cotman C W, Ruehl W W, Milgram N W (1995) Spatial learning and memory as a function of age in the dog. Behavioral Neuroscience 109: 851-858.

Landsberg G, Ruehl W (1997) Geriatric behavioral problems. [Review] [30 refs]. Veterinary Clinics of North America—Small Animal Practice 27: 1537-1559.

Milgram N W, Head E, Weiner E, Thomas E (1994) Cognitive functions and aging in the dog: acquisition of nonspatial visual tasks. Behavioral Neuroscience 108: 57-68.

Milgram, N. W., Zicker, S. C., Head, E., Muggenburg, B. A., Murphey, H., Ikeda-Douglas, C. J., and Cotman, C. W. Dietary enrichment counteracts age-associated cognitive dysfunction in canines. Neurobiology of Aging 23 [5], 737. 2002.

Nielson, J. C., Hart, B. L., Cliff, K. D., Ruehl, W. W., 2001. Prevalence of behavioral changes associated with age-related cognitive cognitive impairment in dogs. J. Am. Vet. Med. Assoc. 218, 1787-1791.

Patel N, Spangler E L, Greig N H, Yu Q S, Ingram D K, Meyer R C (1998) Phenserine, a novel acetylcholinesterase inhibitor, attenuates impaired learning of rats in a 14-unit T-maze induced by blockade of the N—methyl—D—aspartate receptor. Neuroreport 9: 171-176.

Perry E K, Perry R H, Blessed G, Tomlinson B E (1977) Necropsy evidence of central cholinergic deficits in senile dementia. Lancet 1: 189.

Perry E K, Tomlinson B E, Blessed G, Bergmann K, Gibson P H, Perry R H (1978) Correlation of cholinergic abnormalities with senile plaques and mental test scores in senile dementia. B M J 2: 1457-1459.

Ruehl W W, Bruyette D S, DePaoli A, Cotman C W, Head E, Milgram N W, Cummings B J (1995) Canine cognitive dysfunction as a model for human age-related cognitive decline, dementia and Alzheimer's disease: clinical presentation, cognitive testing, pathology and response to 1—deprenyl therapy. pp 217-225. Boulton.

Siwak C T, Tapp P D, Milgram N W (2001) Effect of age and level of cognitive function on spontaneous and exploratory behaviors in the beagle dog. Learning & Memory 8: 317-325.

Su M Y, Head E, Brooks W M, Wang Z, Muggenburg B A, Adam G E, Sutherland R, Cotman C W, Nalcioglu O (1998) Magnetic resonance imaging of anatomic and vascular characteristics in a canine model of human aging. Neurobiology of Aging 19: 479-485.

Tapp P D, Siwak C T, Estrada J, Head E, Muggenburg B A, Cotman C W, Milgram N W (2003a) Size and reversal learning in the beagle dog as a measure of executive function and inhibitory control in aging. Learning & Memory 10: 64-73.

Tapp P D, Siwak C T, Estrada J, Holowachuk D, Milgram N W (2003b) Effects of age on measures of complex working memory span in the beagle dog (Canis familiaris) using two versions of a spatial list learning paradigm. Learning & Memory 10: 148-160.

Whitehouse P J, Price D L, Struble R G, Clark A W, Coyle J T, Delon M R (1982) Alzheimer's disease and senile dementia: loss of neurons in the basal forebrain. Science 215: 1237-1239.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

What is claimed is:

1. A method for treating age related behavioral problems in companion animals comprising: obtaining an effective amount of phenserine; and administering said effective amount of said phenserine to a companion animals for purposes of treating age related behavioral problems in said companion animals.

2. The method for treating age related behavioral problems in companion animals in accordance with claim 1 wherein said phenserine improves learning in said companion animalss.

3. The method for treating age related behavioral problems in companion animals in accordance with claim 1 wherein said phensirine improves trainability in said companion animals.

4. The method for treating age related behavioral problems in companion animals in accordance with claim 1 wherein said treatment with said phenserine improves memory in said companion animals.

5. The method for treating age related behavioral problems in companion animals in accordance with claim 1 wherein said treatment with said phenserine improves clinical signs associated with Canine Cognitive Dysfunction Syndrome.

6. The method for treating age related behavioral problems in companion animals in accordance with claim 1 wherein said treatment with said phenserine improves discrimination learning.

7. The method for treating age related behavioral problems in companion animals in accordance with claim 1 wherein said treatment with said phenserine improves spatial memory.

8. The method for treating age related behavioral problems in companion animals in accordance with claim 3 wherein said treatment with said phenserine improves object recognition memory.

9. The method for treating age related behavioral problems in companion animals in accordance with claim 1 wherein said phenserine is administered at a dose between 0.01 to 1 mg/kg.

10. The method for treating age related behavioral problems in companion animals in accordance with claim 1 wherein said phenserine is administered at a dose between 0.3 and 0.7 mg/kg.

11. The method for treating age related behavioral problems in companion animals in accordance with claim 1 wherein said behavioral problems are associated with beta amyloid protein deposits in said companion animal's brain.

12. The method for treating age related behavioral problems in companion animals in accordance with claim 10 wherein said beta amyloid protein deposits are within frontal, and/or temporal lobes of said brain.

13. The method for treating age related behavioral problems in companion animals in accordance with claim 10 wherein said beta amyloid protein deposits are within prefrontal cortex and/or entorhinal cortex of said brain.

14. The method for treating age related behavioral problems in companion animals in accordance with claim 1 wherein said age related behavioral problems are not linked to any other medical condition, and are associated with learning impairment, memory impairment, or both.

15. The method for treating age related behavioral problems in companion animals in accordance with claim 13 wherein said learning impairment is manifested by difficulty acquiring neuropsychological tasks and decreased trainability.

16. The method for treating age related behavioral problems in companion animals in accordance with claim 13 wherein said memory impairment is manifested by difficulty in performing tasks that include a delay interval.

17. The method for treating age related behavioral problems in companion animals in accordance with claim 1 wherein said age related behavioral problems are associated with a resting electroencephalogram composed predominantly of delta and theta frequencies.

18. The method for treating age related behavioral problems in companion animals in accordance with claim 1 wherein said age related behavioral problems are associated with cholinergic dysfunction.

19. The method for treating age related behavioral problems in companion animals in accordance with claim 1 wherein said age related behavioral problems are associated with cortical atrophy.

20. The method for treating age related behavioral problems in companion animals in accordance with claim 1 wherein said phenserine comprises phenserine analogs in any usable pharmaceutical preparation and/or acceptable salt thereof.

21. The method for treating age related behavioral problems in companion animals in accordance with claim 1 wherein said phenserine comprises phenserine derivatives.

22. The method for treating age related behavioral problems in companion animals in accordance with claim 1 wherein said treatment with said phenserine reduces amyloid protein deposits.

23. The method for treating age related behavioral problems in companion animals in accordance with claim 1 wherein said treatment with said phenserine reduces cortical atrophy.

24. The method for treating age related behavioral problems in companion animals in accordance with claim 1 wherein said treatment with said phenserine increases cholinergic transmission.

25. The method for treating age related behavioral problems in companion animals in accordance with claim 1 wherein said treatment with said phenserine is combined with selegiline.

26. The method for treating age related behavioral problems in companion animals in accordance with claim 1 wherein said treatment with said phenserine is combined with antioxidants.

27. The method for treating age related behavioral problems in companion animals in accordance with claim 1 wherein said treatement with said phenserine is combined with mitochondrial cofactors.

28. The method for treating age related behavioral problems in companion animals in accordance with claim 1 wherein said treatment with said phenserine is combined with DHA.
